(19) United States
(12) Patent Application Publication
(10) Pub. No.: US 2021/0280323 A1
(43) Pub. Date: Sep. 9, 2021

(54) CUSTOMIZABLE COMMUNICATION PLATFORM WITH LONGITUDINAL ALERT TAGS

(71) Applicant: CAREMINDR Corporation, Los Gatos, CA (US)

(72) Inventors: Harry Raymond Soza, San Jose, CA (US); Irina Yermilov, Los Angeles, CA (US)

(21) Appl. No.: 17/156,506

(22) Filed: Jan. 22, 2021

Related U.S. Application Data
(60) Provisional application No. 62/985,846, filed on Mar. 5, 2020.

(51) Int. Cl. G16H 80/00; G16H 20/00; G16H 10/60; G16H 40/67

(52) U.S. Cl. CPC G16H 80/00; G16H 40/67; G16H 10/60; G16H 20/00

(57) ABSTRACT

Processing patient information and other treatment plan information may require access to a patient profile and other third parties involved in the patient treatment plan. One example method includes one or more of, linking a patient device and a healthcare provider server, requesting a patient response to a message, receiving the patient response, determining a content similarity of the patient response to at least one previous response, determining a content recurrence count and a content recurrence timeliness of the patient response, determining an urgency level of the patient response, tagging the patient response as an urgent tagged response if the determined urgency level exceeds a predetermined urgency threshold of the patient, determining a set of possible reasons that the patient response triggered the urgent tagged response and presenting to the healthcare provider device a list of alternative treatment regimens in response to the urgent tagged response.

### BACKGROUND

Conventionally, the approach to providing users with ongoing communications regarding a plan or other repetitive course of action may leave the majority of the work to the user. The smartphone and other personal computing devices are not being properly utilized when offering users with options for maintaining a course of treatment or a set of goals. The lack of action taken by the professional service provider and/or the user can lead to personal health problems and lost revenue for providers, insurers, etc., as well as the users.

### SUMMARY

Example embodiments of the present application provide a first method, comprising, linking a patient device and a healthcare provider server, requesting a patient response to a message sent to the patient device including a query of a health-related issue, receiving the patient response from the patient device to the query, determining a content similarity of the patient response to at least one previous response, determining a content recurrence count of the patient response indicating a similar response content, determining a content recurrence timeliness of the patient response indicating the similar response content, determining an urgency level of the patient response based on the health-related issue, the content recurrence count and the content recurrence timeliness, tagging the patient response as an urgent tagged response if the determined urgency level exceeds a predetermined urgency threshold of the patient for the health-related issue, the content recurrence count and the content recurrence timeliness, providing the request and the urgent tagged response to a healthcare provider device, determining a set of possible reasons that the patient response triggered the urgent tagged response and presenting to the healthcare provider device a list of alternative treatment regimens in response to the urgent tagged response.

A second example non-transitory computer readable medium comprising instructions, that when read by a processor, cause the processor to perform, linking a patient device and a healthcare provider server, requesting a patient response to a message sent to the patient device including a query of a health-related issue, receiving the patient response from the patient device to the query, determining a content similarity of the patient response to at least one previous response, determining a content recurrence count of the patient response indicating a similar response content, determining a content recurrence timeliness of the patient response indicating the similar response content, determining an urgency level of the patient response based on the health-related issue, the content recurrence count and the content recurrence timeliness, tagging the patient response as an urgent tagged response if the determined urgency level exceeds a predetermined urgency threshold of the patient for the health-related issue, the content recurrence count and the content recurrence timeliness, providing the request and the urgent tagged response a healthcare provider device, determining a set of possible reasons that the patient response triggered the urgent tagged response and presenting to the healthcare provider device a list of alternative treatment regimens in response to the urgent tagged response.

A third example embodiment of the present application provides a system, comprising, at least one cloud-based processor and at least one memory electrically coupled to the at least one cloud-based processor and storing an application, wherein the processor performs operations to, link a patient device and a healthcare provider server, request a patient response to a message sent to the patient device including a query of a health-related issue, receive the patient response from the patient device to the query, determine a content similarity of the patient response to at least one previous response, determine a content recurrence count of the patient response indicating a similar response content, determine a content recurrence timeliness of the patient response indicating the similar response content, determine an urgency level of the patient response based on the health-related issue, the content recurrence count and the content recurrence timeliness, tag the patient response as an urgent tagged response if the determined urgency level exceeds a predetermined urgency threshold of the patient for the health-related issue, the content recurrence count and the content recurrence timeliness, provide the request and the urgent tagged response a healthcare provider device, determine a set of possible reasons that the patient response triggered the urgent tagged response and present to the healthcare provider device a list of alternative treatment regimens in response to the urgent tagged response.

### BRIEF DESCRIPTION OF THE DRAWINGS

FIG. 1 illustrates an example of the integrated application platform according to example embodiments.

FIG. 2 illustrates a network configuration of the third party participants of the integrated application according to example embodiments.

FIG. 3 illustrates a user smartphone interface of an example treatment plan according to example embodiments.

FIG. 4A illustrates an example setup configuration for a new course of treatment according to example embodiments.

FIG. 4B illustrates an example database entry for the new course of treatment according to example embodiments.

FIG. 4C illustrates a flow diagram configuration for the new course of treatment according to example embodiments.

FIG. 4D illustrates an example list of messages for the ongoing course of treatment according to example embodiments.

FIG. 4E illustrates an example setup configuration for various courses of treatment according to example embodiments.

FIG. 4F illustrates an example set of details of an ongoing course of treatment according to example embodiments.

FIG. 4G illustrates an example network configuration of the various third parties involved in the application operation and compliance according to example embodiments.

FIG. 5 illustrates a logic module configured to process the input and output parameters of the application according to example embodiments.

FIG. 6 illustrates an example network entity device configured to store instructions, software, and corresponding hardware for executing the same, according to example embodiments of the present application.

FIG. 7 illustrates a data file defining tags and levels of urgency, according to example embodiments of the present application.

FIG. 8 illustrates a patient reply, according to example embodiments of the present application.

FIG. 9 illustrates an electronic report to a healthcare provider with alert tags, according to example embodiments of the present application.

FIG. 10 illustrates system architecture, according to example embodiments of the present application.

FIG. 11 illustrates a first method, according to example embodiments of the present application.

FIG. 12 illustrates a second method, according to example embodiments of the present application.

FIG. 13 illustrates a first non-transitory computer readable medium, according to example embodiments of the present application.

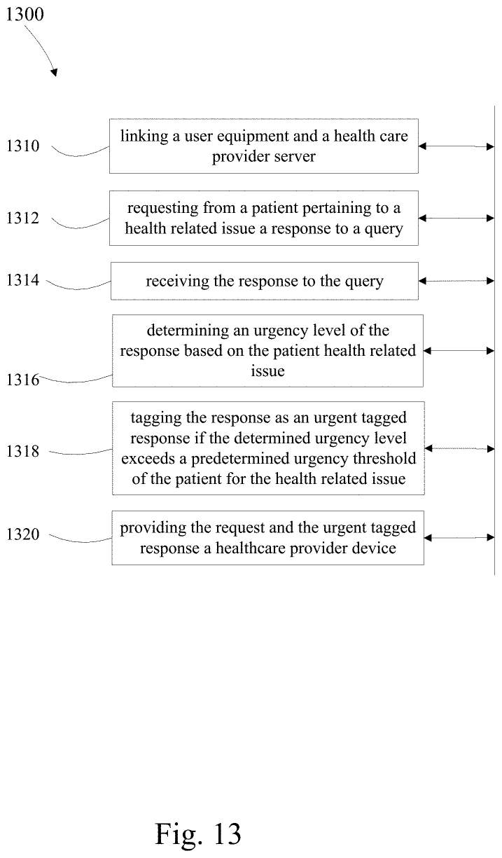

FIG. 14 illustrates a third method of longitudinal alert tagging, according to example embodiments of the present application.

FIG. 15 illustrates a fourth method of longitudinal alert tagging, according to example embodiments of the present application.

FIG. 16 illustrates a fifth method of longitudinal alert tagging, according to example embodiments of the present application.

FIG. 17 illustrates an example multiple measurement warning, according to example embodiments of the present application.

### DETAILED DESCRIPTION

It will be readily understood that the components of the present application, as generally described and illustrated in the figures herein, may be arranged and designed in a wide variety of different configurations. Thus, the following detailed description of the embodiments of a method, apparatus, and system, as represented in the attached figures, is not intended to limit the scope of the application as claimed, but is merely representative of selected embodiments of the application.

The features, structures, or characteristics of the application described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present application. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

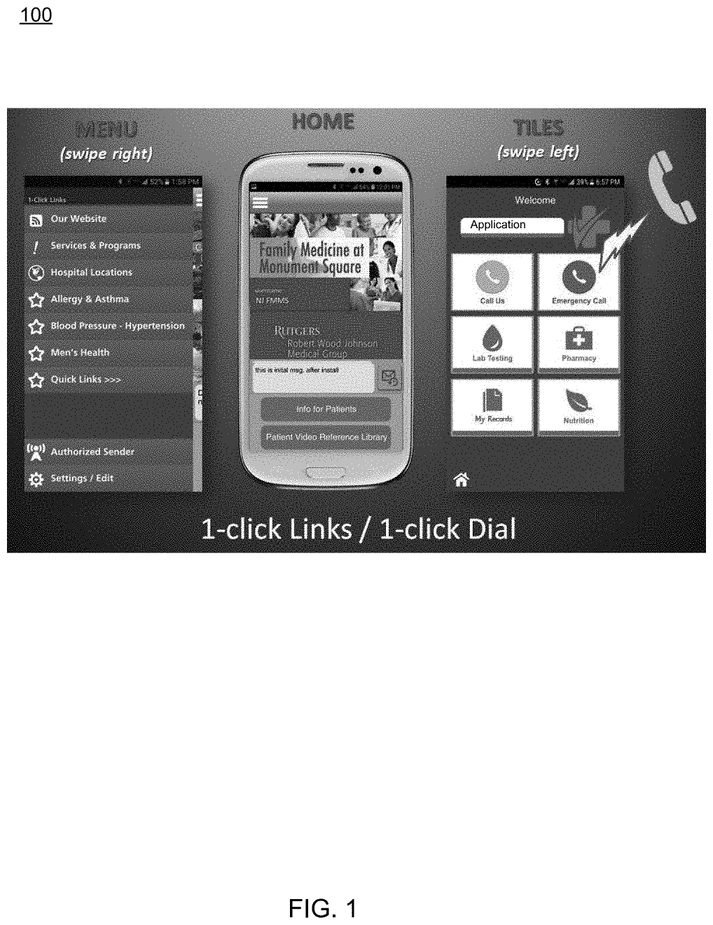

FIG. 1 illustrates an example of the integrated application platform according to example embodiments. Referring to FIG. 1, the configuration 100 includes a menu user interface, a home user interface and a set of option tiles for accessing third party resources, such as test results, emergency concerns, pharmacy information, etc. The tiles may initially be provided by the cloud system and may serve as a conduit of previous learning pertaining to a specific user's condition, treatment plan and schedule. The tiles may be added to by healthcare professionals, in which case the cloud system will include the additional information to perform additional linkages between conditions, treatment plans, drugs, timings and the like. The tiles have within them not just the specific item, but the interlinkages that make insight into specific outcomes much more insightful and make the computer outputs much more targeted.

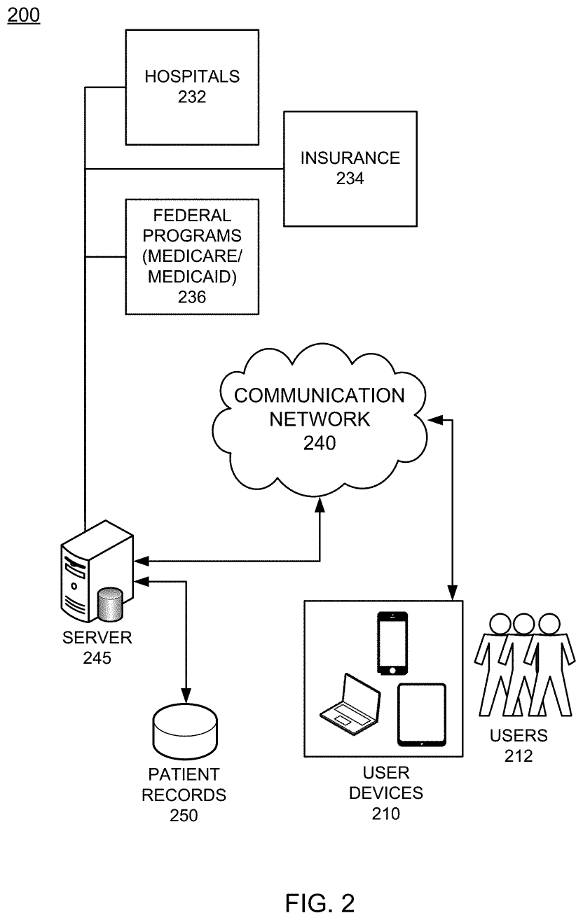

FIG. 2 illustrates a network configuration of the third party participants of the integrated application according to example embodiments. Referring to FIG. 2, the network includes a central server 245 with patient records 250. The information needed to provide treatment plans and other integrated services may require access to hospital and other provider services 232, insurance company information 234, drug providers, federal program administrators 236, etc. The information may be incorporated into any treatment plan or other integrated service model accessed by a user device 210 operated by a user 212. The servers and third party modules may operate on-site or in a cloud network managed by the providers.

Examples of treatment plans and other objectives may include a care management service for assessment of patient medical needs. The system and application may ensure timely receipt of all recommended treatment actions, drugs, third party services over a designated period of time. Also, referrals to other providers and additional services may provide emergency visits, discharge instructions, nursing facility operations, and home healthcare functions. In operation, the procedure may begin with the medical treatment provider creating a treatment plan or ‘journey’ for each patient. Each journey is generally for a single chronic condition or objective. One patient may have multiple journeys integrated into a single application. Also, the journeys may originate from various different providers and service entities. The journey will provide the healthcare provider with biometric, objective and subjective data to enable evidence-based medical decisions. As an example, the biometric data may be glucometer data collected from a blue tooth enabled device and made available to the physician, objective data such as whether the patient visited an emergency room or hospital and subjective data such as how the patient is feeling.

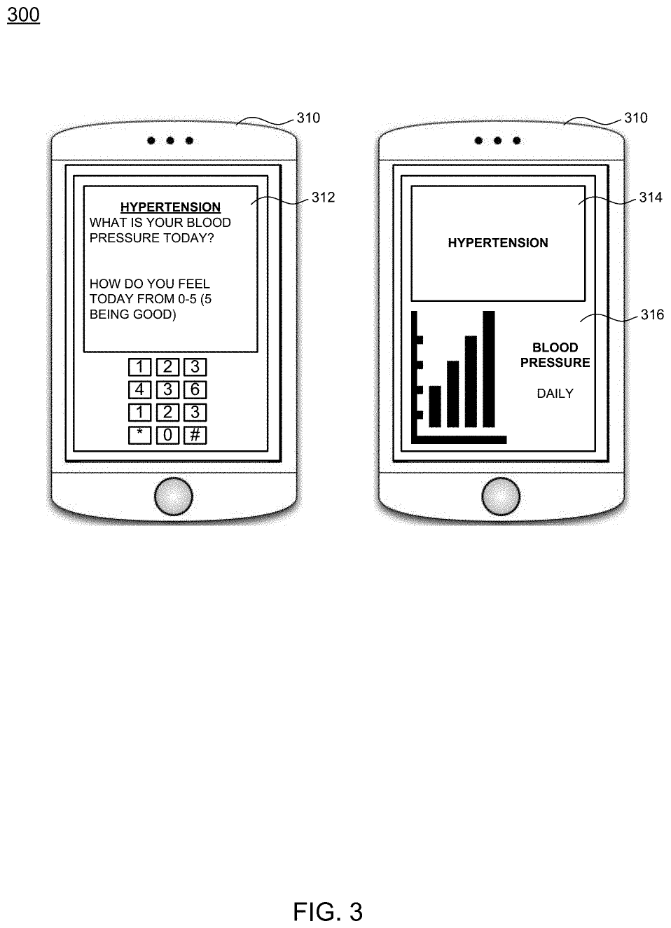

FIG. 3 illustrates a user smartphone interface of an example treatment plan according to example embodiments. Referring to FIG. 3, the journey for “hypertension” may have been created or modified by a patient doctor and may include an interface 300 with a smartphone device 310 and a screen option configuration providing questions 312, information about the treatment, reminders and other functions. The example in FIG. 3 provides for a set of questions 312 and a journey topic 314 along with a graph of blood pressure records 316 as measured over time from various interactions. The system has the capability to connect the application on the patient device to a machine, via a wireless connection such as Bluetooth, such that it may provide medical information directly to the application. Utilizing this capability, the patent device may receive a current reading such as temperature, blood pressure, heart rate, weight, etc., and have that information dynamically provided to the application which may use that information to provide immediate feedback and recommendations.

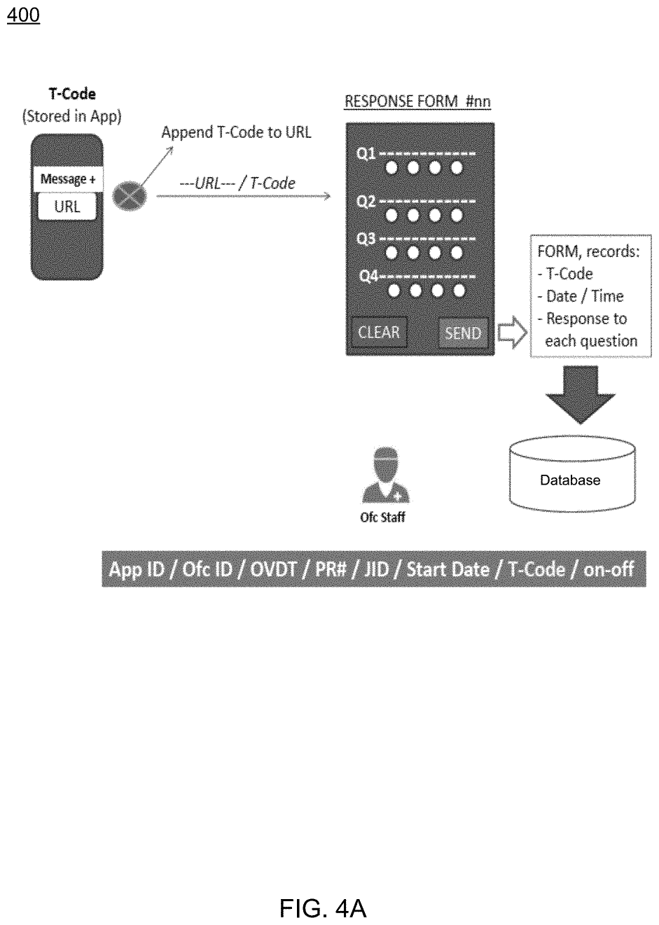

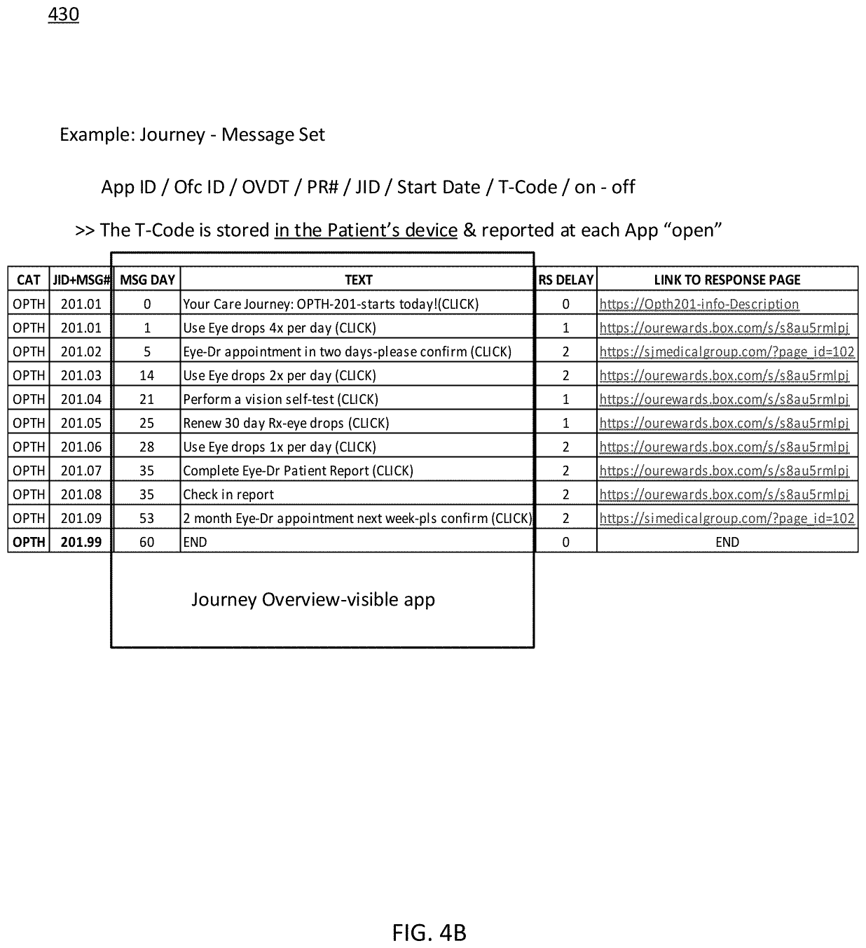

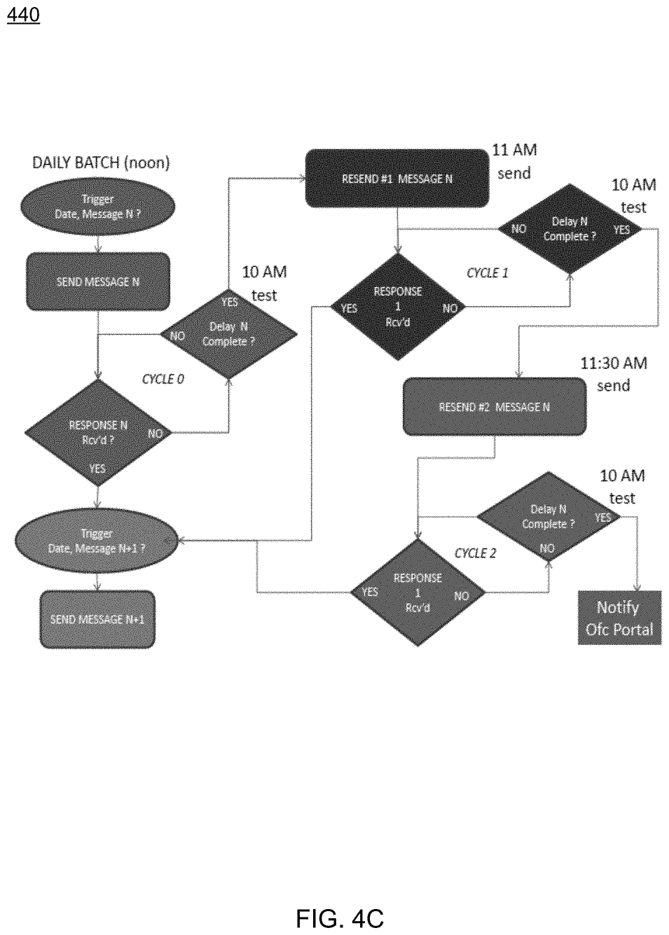

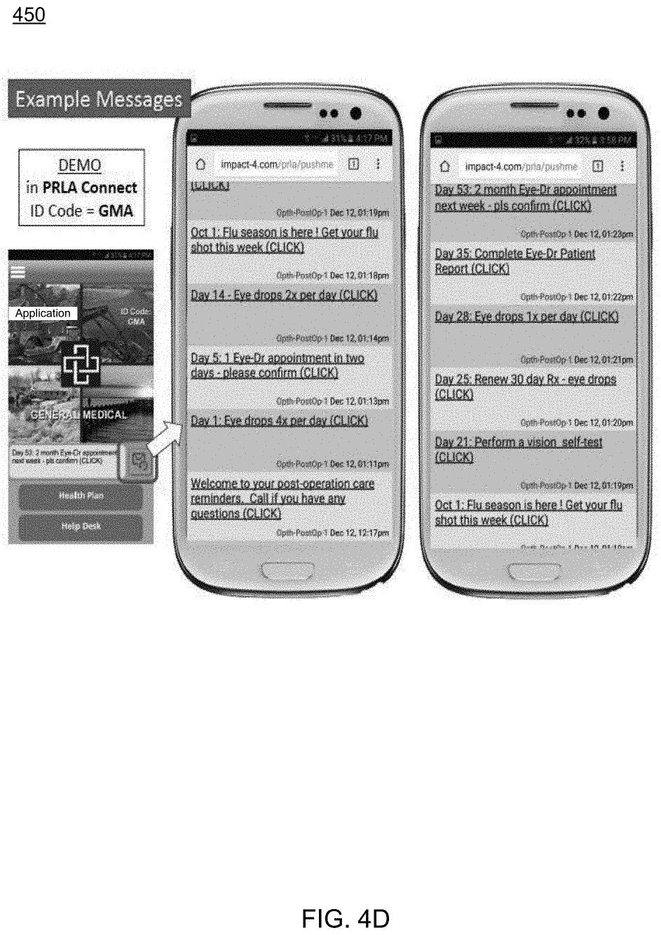

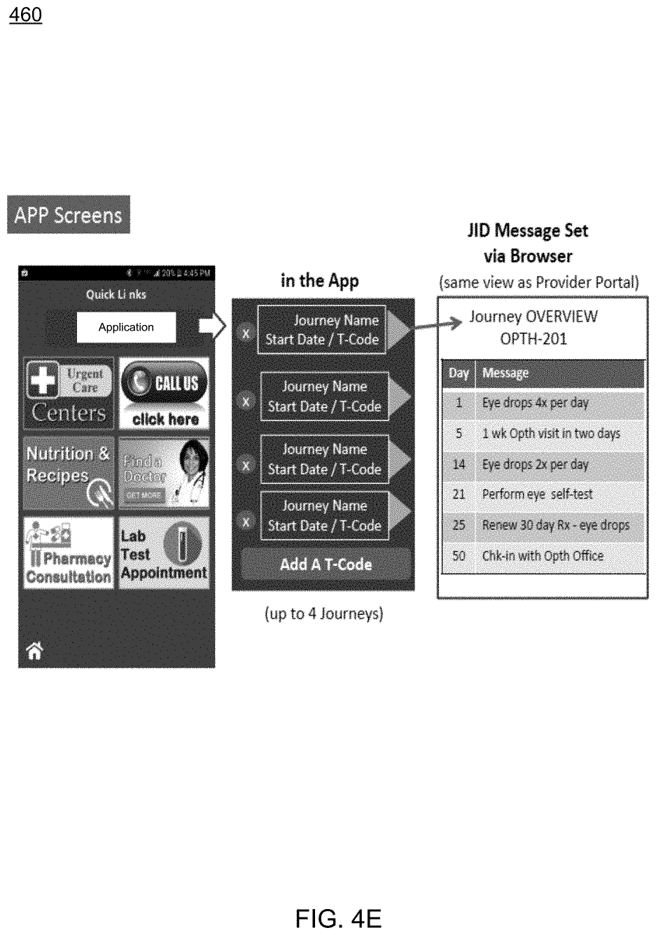

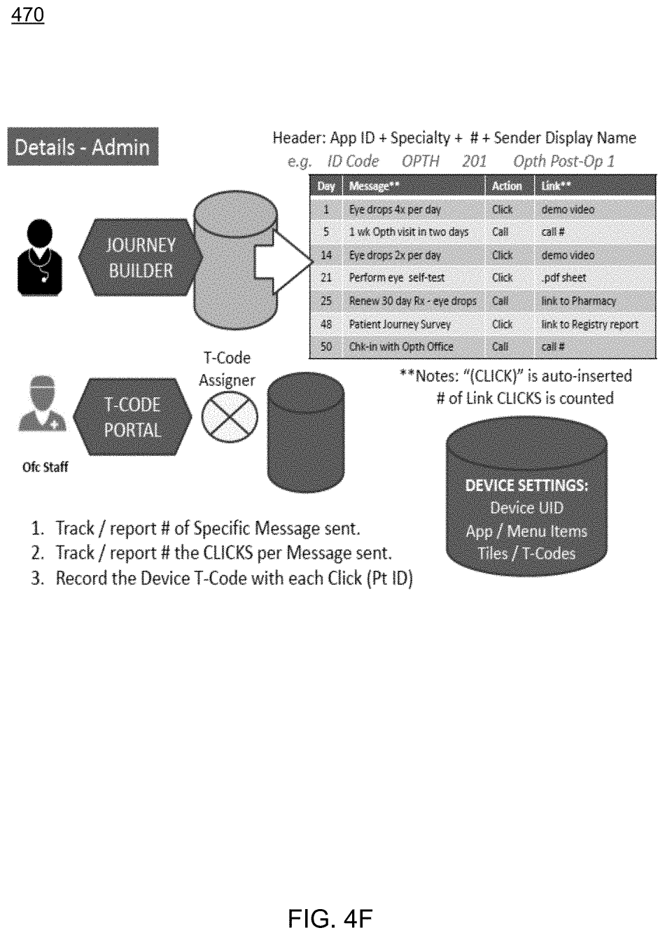

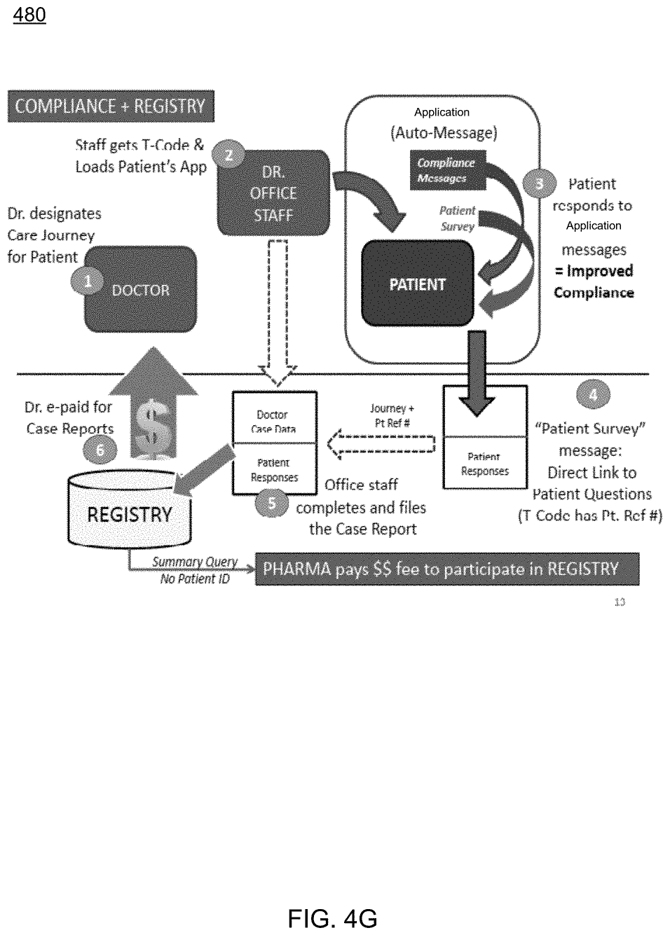

FIG. 4A illustrates an example setup configuration for a new course of treatment according to example embodiments. Referring to FIG. 4A, the illustration 400 includes the basic setup functions of linking a particular journey T-code (unique code) to the message and/or universal resource locator (URL) to link the application of the user device to a customized template, such as a response form, questionnaire, etc. The unique T-code, date, time, response, and other records for each instance may be stored in a patient record managed by the application system database. A T-code means return to provider (RTP) which indicates that a claim has reached its final disposition with no reimbursement due to billing errors. At the bottom of 4A an App ID is the applicant identification, the OVDT is the office visit date and time, PR# is the patient reply number and JID is the journey identification number. The database uses these identifiers to identify patients and isolate which forms to utilize. The form is dynamically filled out in the cloud server 245 with information from the patient records 250.

FIG. 4B illustrates an example database entry for the new course of treatment according to example embodiments. Referring to FIG. 4B, the example configuration 430 includes a database entry of messages which are organized by a category, in this case ophthalmology, and with a message content, including a link to a response page. The context and add-ons of a particular message may be customized based on a preferred layout or a default layout. The messages may be categorized in multiple ways by the cloud server, by patient, by journey, by response, by treatment, by drug, by drug interaction, and the like, in this way interactions of various kinds may be grouped by the system to potentially find commonalities, similarities, beneficial effects, side effects, etc. The response page may be similarly chosen by the cloud server based upon the initial journey or one of the groupings. The search by the cloud based server for similarities and interactions may allow the system to continuously learn from the patients what is the most favorable treatment and what common issues arrive and how those issues are dealt with for other patients having similar demographics and responses. The healthcare professional may also customize the interaction with the patient and the response of the system to certain criteria which he indicates.

FIG. 4C illustrates a flow diagram 440 configuration for the new course of treatment according to example embodiments. Referring to FIG. 4C, the flow diagram includes a daily batch of messages which are set up to be delivered to one or more devices associated with one or more assigned patients. The process begins with a trigger to send a message, such as a matured date or time. The process then continues to deliver additional messages once confirmation of delivery is made. If the message is delayed or the response required is not received, the message may be resent as a late message requiring immediate attention. The process may continue to cycle to identify whether any messages are outstanding or have not been confirmed. The system may track information here related to the type of illness/issue, treatment program, monitoring/effectiveness of a patient and tag according to responses and non-responses of the patient. The system may change tactics if the patient is unresponsive or answers responses indicating issues associated with the treatment journey. The system may change what questions are asked or the frequency of messaging based on the patient response or non-response. In the situation in which a patient is not responding, the system may have the healthcare provider initiate a call directly to the patient.

FIG. 4D illustrates an example list of messages for the ongoing course of treatment according to example embodiments. Referring to FIG. 4D, in this illustration 450, the various messages intended for a particular patient are illustrated by date. The messages may originate at the cloud based system and may be originally designed into the specific journey. The timing and response criteria may be set by the journey and may be modified by the healthcare professional.

FIG. 4E illustrates an example setup configuration for various courses of treatment according to example embodiments. Referring to FIG. 4E, the configuration 460 includes a menu of options along with a set of potential journeys the user device may be assigned to manage the ongoing healthcare treatment plans. The overview of treatment options and dates are included for reference purposes. The patient may be interacting with the system for multiple journeys simultaneously. Each journey may interact with the patient according to its own logical construct. The messages to be sent may originate at the cloud based server and the data received may be received by both the cloud based server and the healthcare provider server. In the cloud server the data is sorted according to patient demographics and interactions, in the healthcare provider server the data is sorted according to patient and healthcare provider. The logic governing the interactions may be controlled by the cloud based server and may be initially set by the specific journey.

FIG. 4F illustrates an example set of details of an ongoing course of treatment according to example embodiments. Referring to FIG. 4F, the details of the administrator are shown to include a journey builder function based on certain parameters, such as an identification code, specialty, a number and a sender name. The number of messages, responses and actions are recorded to demonstrate interaction with the application and the specific treatment plan(s). The journey builder utilizes a spreadsheet style input that has a day, the message to send to the patient, the expected response and a link for the patient to link to. The system tracks the interactions between the sent messages and the patient responses. The patient device is tracked by a device universal identification (UID) and the application and menu items, the selection tiles and the T-codes. The system tracks and reports the number of specific messages sent to the patient, the number of clicks the patient performs and records the device T-code with each click in association with the patient identification number (Pt ID).

FIG. 4G illustrates an example network configuration of the various third parties involved in the application operation and compliance according to example embodiments. Referring to FIG. 4G, the network of communications among the integrated platform 480 demonstrates the process initiating with the doctor's office establishing a journey for the patient and assigning a T-code (unique code). The patient's responses are identified along with links and references to third party message links and other information sources. The doctor designates a journey for the patient based on the patient's condition and the treatment plan. The healthcare providers staff gets a T-code for the treatment plan and sends the application to the patients device. The journey application sends messages to the patient, receives responses to surveys and assesses compliance to the journey protocols. The patient responses to the patent survey messages directly link the response to the query for that patient. The journey sends the data to the cloud server in scrubbed form and sends the data to the healthcare providers server linking the patient reference number to the journey. The doctor's case files and the patient responses are compiled into a case report that is sent to a registry.

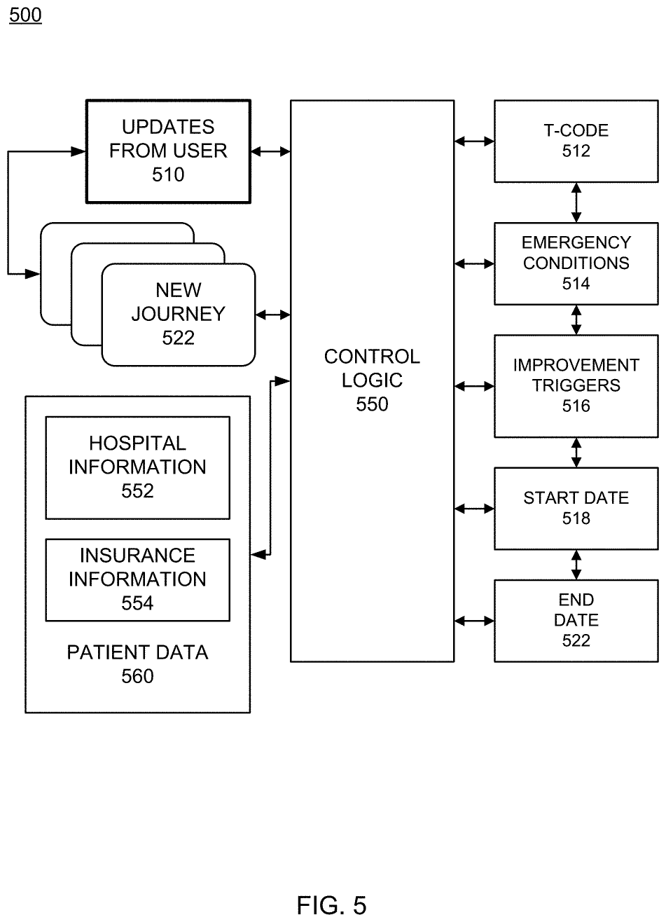

FIG. 5 illustrates a logic module configured to process the input and output parameters of the instant application according to example embodiments. Referring to FIG. 5, the control logic platform 500 includes a control logic unit 550, such as a processor or other processing entity that may receive updates from a user 510, such as by keyboard, voice processing and the like, new journey information 522 and/or patient data 560 including hospital 552, insurance 554, doctor, office, prescriptions and the like. The logic may be configured to identify and link the unique T-code 512, emergency conditions 514, improvement triggers 516 for optimal changes to the treatment plan, along with dates 518 and new journey information 522 to perform the treatment plan.

In addition, while the term “message” has been used in the description of embodiments of the present application, the application may be applied to many types of network data, such as, packet, frame, datagram, etc. For purposes of this application, the term “message” also includes packet, frame, datagram, and any equivalents thereof. Furthermore, while certain types of messages and signaling are depicted in exemplary embodiments of the application, the application is not limited to a certain type of message, and the application is not limited to a certain type of signaling.

According to example embodiments, a user device, such as a smartphone, cellular phone, tablet device, laptop or other computing device with a memory and processor, may communicate with another computing device and/or a server to provide an integrated communication platform.

Example embodiments provide a computer system programmed to use automated messaging from medical offices to specific patients. The application is not limited to medical procedures and functions and may be used with other configurations for various purposes and services benefitting the end user. Example embodiments include three main computer systems, which work together in an integrated manner including a management platform that controls set-up, functionality, activity reporting, and messaging credentials for the users. An administrative platform which the doctor and doctor's office can access via the internet, and a mobile application that a patient can download into a mobile computer device such as a smartphone or tablet. The management platform acts as the nexus of the system sending outgoing messages on behalf of the healthcare provider and forwarding patient responses to the healthcare provider's administrative platform. The medical office may have a specific identification that is stored within the management platform.

The integrated platform provides a way of checking-in with a patient at prescribed intervals during times between office visits and when undergoing certain treatment that the doctor is providing or overseeing for the patient. The patient dialog may gather relevant information about the status of the patient's conditions or recovery and can be modified or tailored to specifically meet the dialog requirements of the treating physician. Once initiated by the doctor's office, the application operates in an autonomous manner by delivering messages to the patient to prompt responses if needed. The application functions are monitored to assure that the patient replies to the information requests from the doctor, otherwise a no-response alert is sent to the doctor's office. The interactions are recorded and time-stamped, providing an auditable record of the dialog, suitable for insurance billing purposes. The application can also support biometric information from devices that measure certain body functions, such as diabetes glucometers, or blood pressure cuffs, or any sensory readable healthcare metric. The application may also create a longitudinal record of information for the patient to illustrate week-to-week trends.

### Response Alert Tags

As has been stated earlier, this method and system is utilized when a patient visits a healthcare provider for an illness/condition which is diagnosed and treated. The treatment occurs over a period of time and is referred to as a journey. The system tracks a patient's progress along the journey for that illness or condition and solicits health information from the patient at clinically-relevant intervals, across an extended time period to enable evidence based medicine. The specific information sought, the intervals, and the time period duration apply to specific conditions or illnesses for which a specific patient is being treated.

This solicitation for patient information from the cloud server may take the form of queries sent to the patient via the patient device for information, when the responses to those queries are delivered to the patient's healthcare provider server to the physician. The patient's journey may have a number of waypoints occurring at the clinically-relevant intervals. The responses to the queries from the patient device at these waypoints are meant to determine a patient's progress and status and to present to the healthcare provider evidence upon which to conduct evidence-based medicine. The responses are collected by the system and measured against historical norms for the patient and/or expected answers for similar patients on similar journeys.

In the event of an unexpected response to a query at that waypoint, the response is treated as notable. Notable events may be considered non-urgent or may be considered urgent or emergent. This divergence from the expected response outcome is graded for severity or urgency. If the severity or urgency of the response exceeds a predetermined threshold for that patient for that journey for that illness or condition at that waypoint, an urgent tag is created and sent to the healthcare provider. The grading may be one of an immediate medical action advisory, a follow-up advisory and a medical history review advisory.

The information requested in the query is sent in a structured format to allow ease in answering and the response data is delivered to the healthcare provider in a structured data format to facilitate ease in analysis and trend detection.

The response alert tag is a feature that tags certain responses provided by the patient via the patient device as information that requires follow-up or special notice sent to the healthcare provider device. The tag may indicate a level of severity or urgency, thus alerting the provider to information that may need immediate medical action, additional follow-up with the patient or a specific review of the patient's medical history. The tag settings are set at the cloud server and the alert tags are sent by the cloud server.

The tag may be communicated to the provider through multiple channels depending upon circumstance and urgency and in an immediate manner or in a weekly aggregated format depending in part upon urgency.

Work flow instructions may be electronically linked to a tag, so that the specific healthcare provider that reviews the data will have guidance about the actions to be taken when a tag appears and any escalation of clinical review that might be appropriate.

Each patient for each illness or condition is interacted with by the system at intervals which are relevant to that illness or condition and the queries are sent to determine the patient's progress or status. The received response to the query is measured against an expected response, and anomalies or offsets are noted. If these response anomalies or offsets are larger than a predetermined amount, an urgent or severe issue may need to be addressed. Thus the response is tagged as an urgent tagged response and may be sent utilizing a priority delivery schedule, a priority delivery indicia on the response and may be made to a priority delivery list determined by the healthcare provider. The response may be tagged as non-urgent if the determined urgency level does not meet the predetermined urgency threshold of the patient for the health related issue.

The structured format allows an overlap of queries so that the patient is not answering multiple identical queries at any one point in time. Additionally, the structured format allows the data to be collected and logged in a structured format and assembled for future review both by the practitioner and the patient to determine trends.

In one example, a method includes requesting via a cloud based system from a patient response to a query and receiving the response to the health related query, determining an urgency level of the response by the cloud based server, based on the patient health related issue and tagging by the cloud based server the response as urgent if the determined urgency level exceeds a predetermined urgency threshold of the patient for the health related issue.

The method may also include providing the urgent tagged response to the health provider by the cloud based server, where the urgent tagged response may be sent from the cloud based server utilizing a priority delivery schedule, a priority delivery indicia for the response and may be made to a priority delivery list. The output of the tagged responses may also be prioritized by urgency.

The method may also include tagging by the cloud based server the response as non-urgent if the determined urgency level does not meet the predetermined urgency threshold of the patient for the health related issue.

If the determined urgency level of the response is such that it rises to the level of a medical emergency, then the primary care physician may be immediately notified as well as emergency services such as 911 and if deemed appropriate, dispatched to the location identified either by the patient or gathered from a location indicator in his mobile device. The urgency level determination may be performed by the cloud based server. If the response is deemed critical, in situations where the primary physician is not immediately available, an emergency medical specialist may be placed in active direct communication with the patient. The system would make available to the first responder the query and response to provide context for the escalation.

The response may be graded as to the tagged urgency level of the response, where the grading is at least one of an immediate medical action advisory, a follow-up advisory and a medical history review advisory. A follow-on query may be sent based on the urgent tagged response to give the provider context to the urgent tagged response. As an example, if the patient responds that they have been to the emergency room (ER) that may trigger another set of queries about the ER visit to add context to the response. This second set of queries may determine whether the ER visit was related to conditions or illnesses related to the journey, or whether visit was for a condition unrelated to the journey, but still of interest to the healthcare provider.

In another example a cloud based system links patient device and a healthcare provider server. The cloud based system requests a response to a query from a patient device of a patient pertaining to a health related issue, receives the response to the query via the patient device and determines an urgency level of the response based on the patient health related issue. The system also tags the response as an urgent tagged response if the determined urgency level exceeds a predetermined urgency threshold of the patient for the health related issue and provides the urgent tagged response to a health provider device.

The cloud based system may receive via the patient device a sensor signal provided by a medical device in response to the query. The medical device may be a blood pressure monitor, a glucometer, a pulse meter, a continuous positive airway pressure device, a heart monitor, an implanted medical device and the like.

The cloud based system may receive via the patient device an audio or text message indicating a medical distress condition in response to the query or may overhear the patient indicating a medical distress condition in conversations or texts in an unsolicited message.

The system may also interpret patient actions in regards to patient historical norms, such as, if the patient is overheard slurring his speech, he may be having a stroke, or if he is discussing that he has pressure in his chest or his left arm is numb, he may be having a heart attack. At this point the system may connect the patient device directly to a medical specialist and take other appropriate action, such as determining the patient device location and dispatching emergency services.

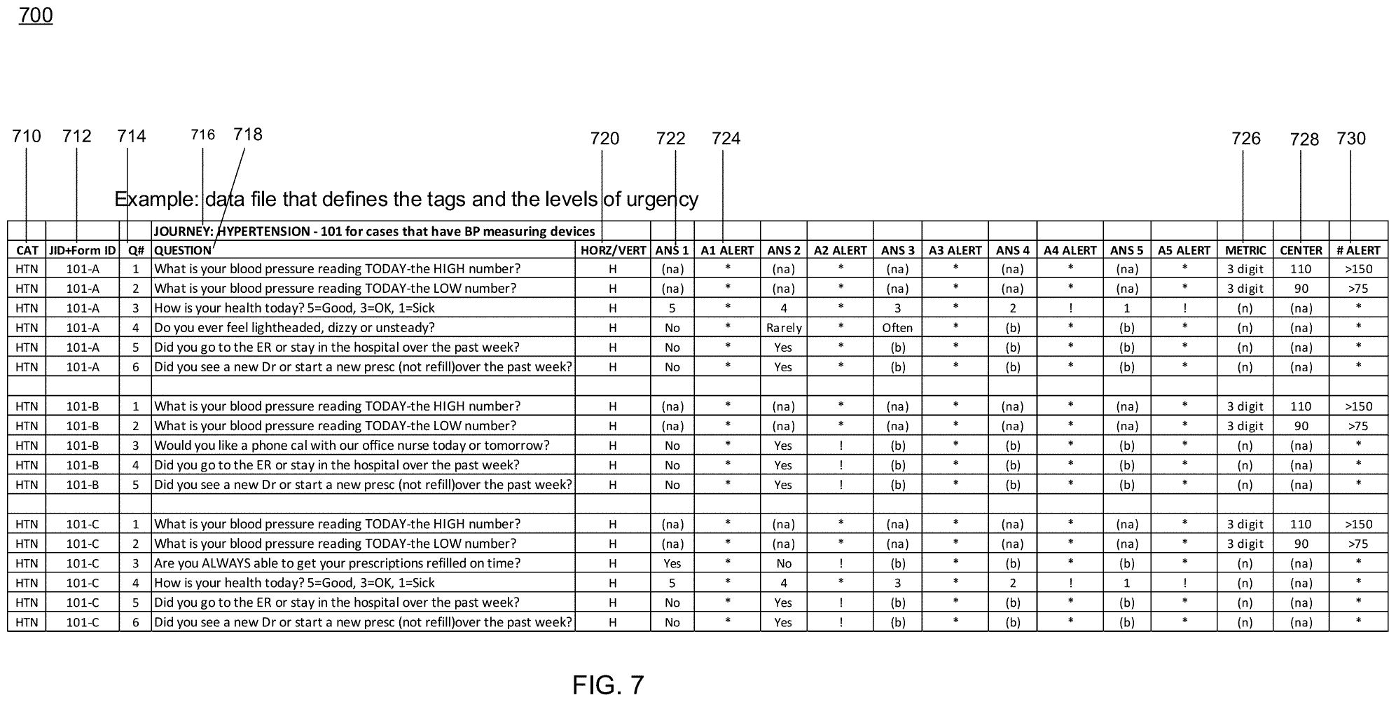

FIG. 7 depicts an example data file 700 that defines the tags and levels of urgency. The parts of the data file include a category of question 710, the journey ID and form ID 712, a question number 714, a patient journey for that specific illness or condition 716 and questions associated with that journey 718. The data layouts may be shown horizontally or vertically 720 which allows viewing of individual answers to queries. The historical and current query response 722 and the determined alert 724 are shown, which in a historical context allows detection of trends. The data output of the metric 726 may be numerical, yes or no, and the like, a median query answer 728 for that patient or that condition and an alert threshold 730 which may be modified by the healthcare profession are shown.

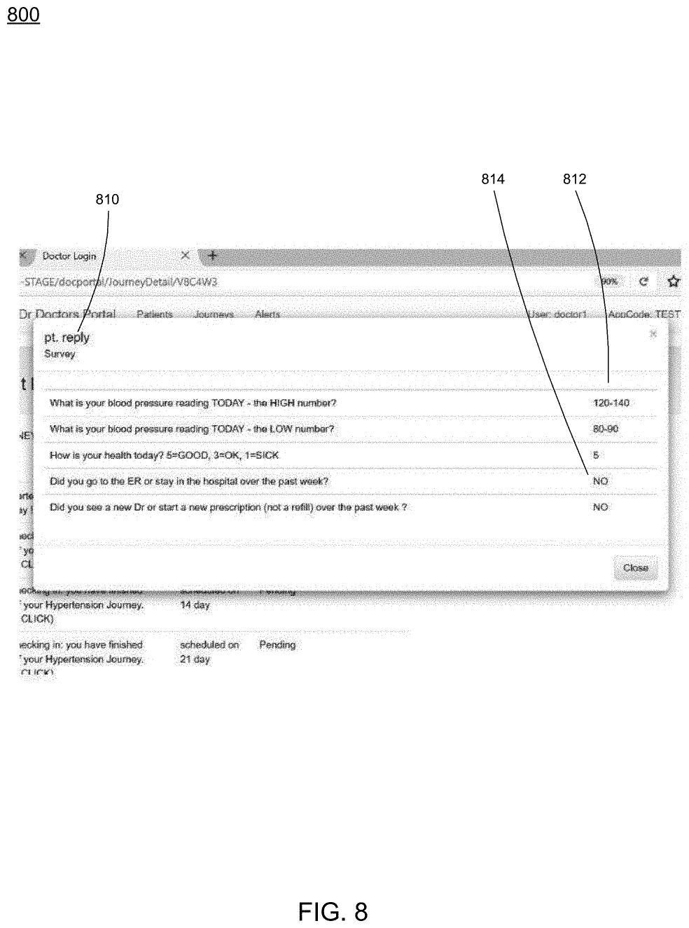

FIG. 8 depicts a patient survey reply 810 having a band of expected replies for blood pressure 812, how the patient feels and whether he went to the emergency room, hospital 814 or has started a new prescription.

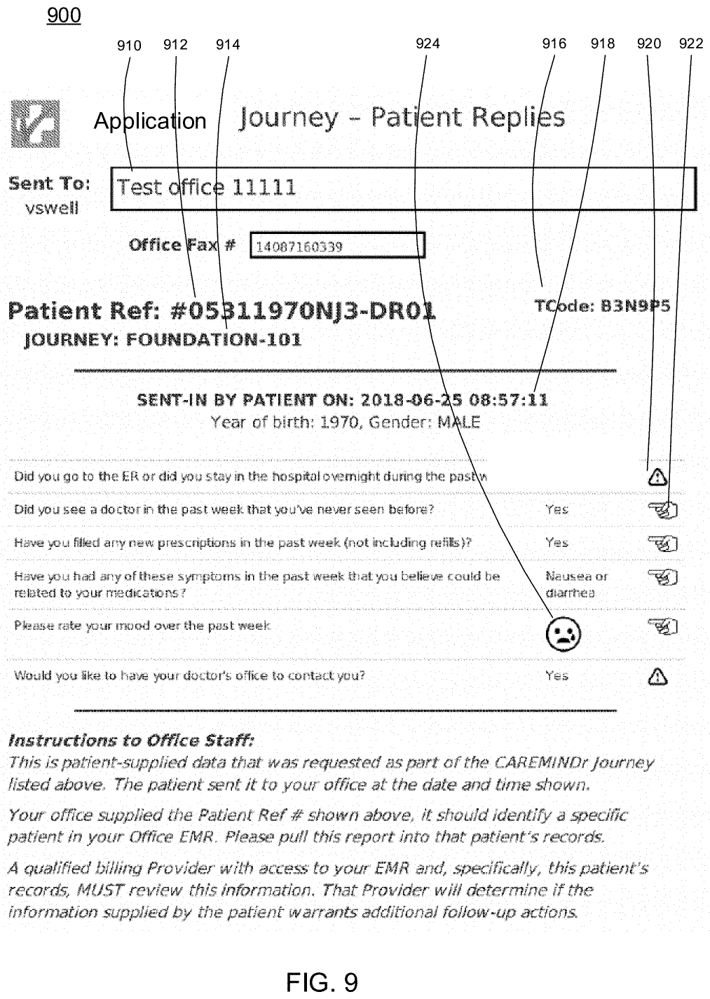

FIG. 9 depicts both a non-responsive and responsive reply set 900. The recipient 910, patient reference number 912, journey ID 914, unique T-Code 916 and timestamp 918 are depicted in the reply. A responsive report having alerts indicates an urgent issue 920, a follow up item 922 and an emoticon 924 indicating a patient feeling is shown.

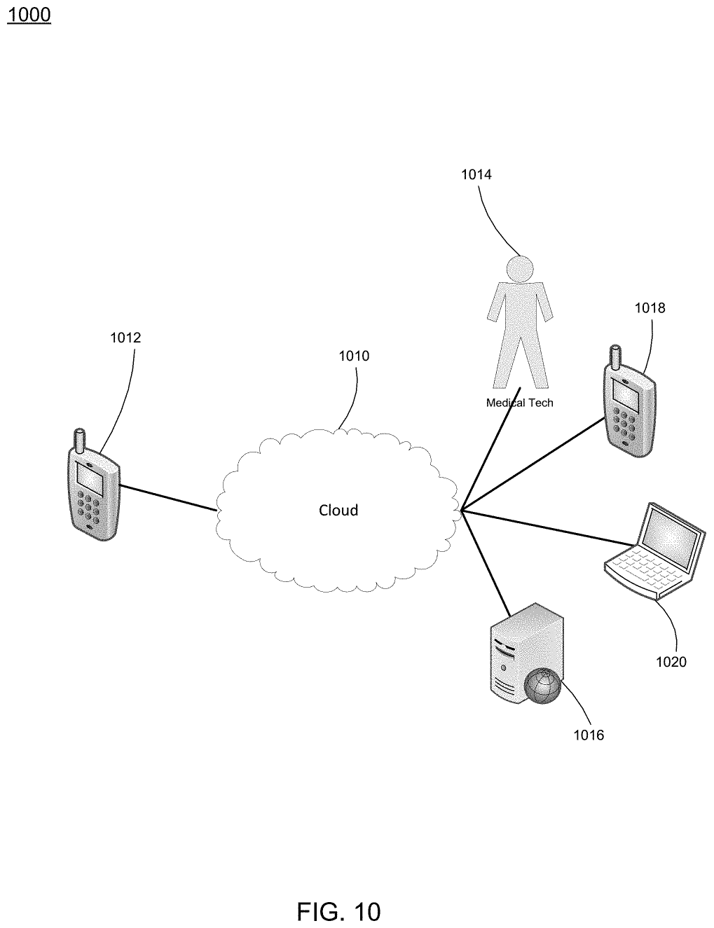

FIG. 10 depicts an example 1000 of communication associated with the alert tag. The cloud based system 1010 sends a request with queries to the patient's communication device 1012 which the patient fills out and returns. The patient's communication device may also be termed the patient device herein. The cloud based system 1010 reviews the response from the patient device and determines whether there are urgent or emergency issues and sends an urgent tagged response to the healthcare provider device.

If there is an emergency issue the cloud based system may contact the patient device or place the patient in contact with a medical technician 1014 in addition to notifying the healthcare provider device by means of the healthcare provider's server 1016, the cloud based system may issue a text or message to the healthcare provider device. The communication route from the healthcare provider device may be by means of mobile device 1018, computer 1020 or the like. The cloud based system may directly connect the patient via to the patient's communication device 1012 to the healthcare provider device under appropriate circumstances. Non-urgent issues are sent to the healthcare provider server for later review.

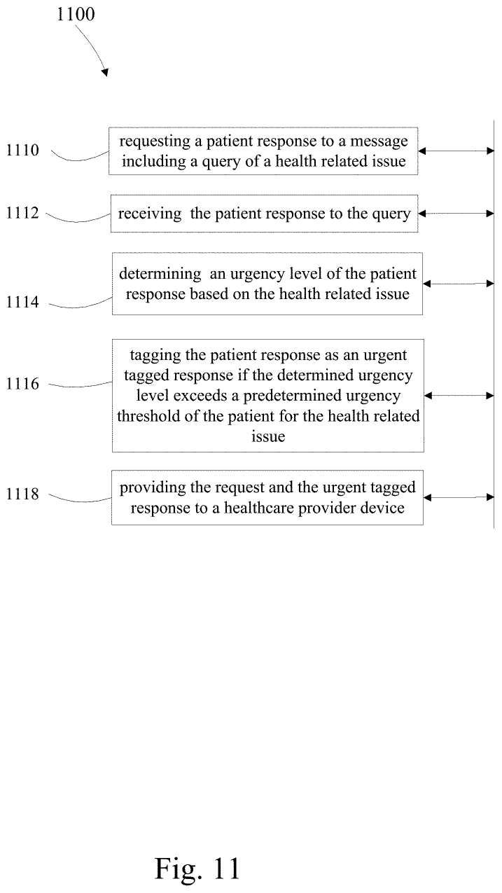

A second example method is shown in FIG. 11 related to response tagging, may include requesting 1110 a patient response to a message including a query of a health related issue and receiving 1112 the patient response to the query from the patient device. The method then provides determining 1114 an urgency level of the patient response based on the health related issue, tagging 1116 the patient response as an urgent tagged response if the determined urgency level exceeds a predetermined urgency threshold of the patient for the health related issue and providing 1118 the request and the urgent tagged response to a healthcare provider device. The method may also include proposing a set of work flow instructions linked to an urgent or non-urgent tagged response and presenting a clinical escalation review advisory to the healthcare provider server and the healthcare provider device if the response is tagged as an urgent tagged response.

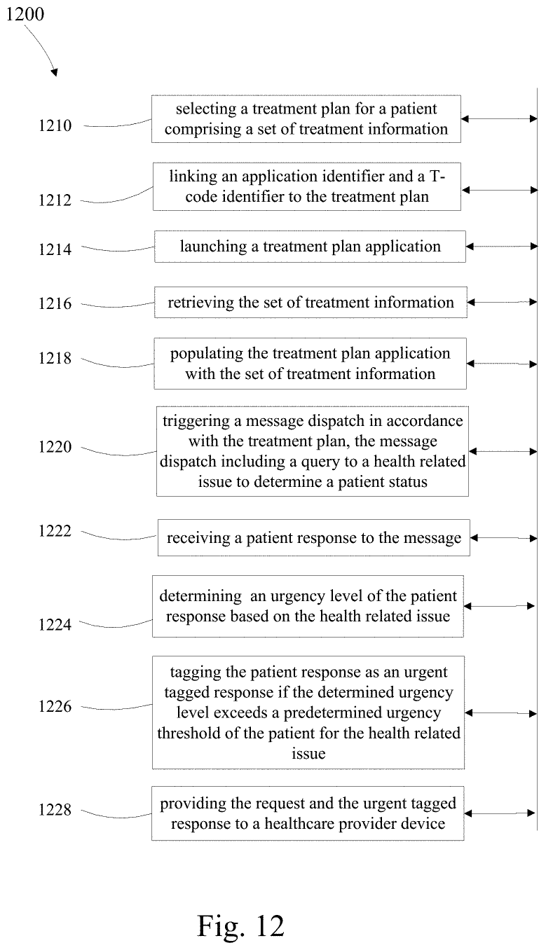

A first example method shown in FIG. 12, 1200, may include, selecting 1210 a treatment plan for a patient comprising a set of treatment information, linking 1212 an application identifier and a T-code identifier to the treatment plan and launching 1214 a treatment plan application. The method further includes retrieving 1216 the set of treatment information, populating 1218 the treatment plan application with the set of treatment information and triggering 1220 a message dispatch in accordance with the treatment plan. The message dispatch includes a query to a health related issue to determine a patient status and the system receives 1222 a patient response to the message from the patient device. The method then provides determining 1224 an urgency level of the patient response based on the health related issue, tagging 1226 the patient response as an urgent tagged response if the determined urgency level exceeds a predetermined urgency threshold of the patient for the health related issue and providing 1228 the request and the urgent tagged response to a healthcare provider device. The method may also include proposing a set of work flow instructions linked to an urgent or non-urgent tagged response and presenting a clinical escalation review advisory if the response is tagged as an urgent tagged response.

With respect to the timing of patient responses, the first example method may also include, awaiting the patient response from the patient device to the message for a late response duration and categorizing the patient response if the patient response is received within the late response duration. If the patient response is not received within the late response duration the method further comprises sending a duplicate message and flagging the patient response as non-responsive if the patient response to the duplicate message is not received within a second late response duration from the patient device.

The timing of the message dispatches associated with the treatment plan is partly governed by a trigger table. The method may include loading the trigger table having a set of trigger dates based on the treatment plan where the message dispatch is sent according to the set of trigger dates. The method may further include receiving a message start date and receiving an initialization message from a patient mobile device to initiate the treatment plan and to initialize the set of trigger dates in the trigger table.

A first example non-transitory computer readable medium 1300 comprising instructions associated with the tagging of responses that, when read by a processor, cause the processor to perform; linking 1310 a patient device and a healthcare provider server, requesting 1312 from a patient pertaining to a health related issue a response to a query and receiving 1314 the response to the query. The processor then determines 1316 an urgency level of the response based on the patient health related issue, tags 1318 the response as an urgent tagged response if the determined urgency level exceeds a predetermined urgency threshold of the patient for the health related issue and provides 1320 the request and the urgent tagged response a healthcare provider device.

### Longitudinal Response Alert

The system solicits healthcare-related information from a patient device based on healthcare-related information requests and delivers that information to that patient's healthcare provider server and/or healthcare provider device. The information that is requested may be in a structured format that includes a data set of patient responses sent from the patient device. The requests for healthcare-related information by a cloud-based server or healthcare-provider server may occur at clinically-relevant intervals, across an extended time period. The specific information, the intervals, and the time period duration may be related to specific conditions or illnesses for which a specific patient is being treated.

One potential solution that may be provided is that the cloud server or healthcare provider server may have the ability to tag certain patient responses from the patient device as relevant for further review.

The tag assigned by the cloud server or healthcare provider server may indicate levels of severity or urgency alerting the healthcare provider device of information that may need immediate medical action or follow-up.

This solution may addresses a subtle situation where a one-time response from a patient device is not designated urgent and does not warrant immediate action, but a recurring pattern of this same response may be determined to be urgent and may constitute a more serious issue. The medical condition, the treatment regimen, the history of patient responses, and the timeline of recurrence of similar patient responses, taken together, may create be determined to constitute a higher urgency by the cloud server or healthcare provider server a singular response.

In one example, a patient response from a patient device pertaining to a new medication may indicate the one time feeling light-headedness, which may be a common side effect to starting that medication. In this situation, the cloud server or healthcare server may determine that no action is warranted. However, if the responses from the patient device continue to report this symptom for 5 days, then a follow-up action may be scheduled by the cloud server or healthcare provider server.

This longitudinal tag may be communicated to the healthcare provider through the cloud server or healthcare provider server to a healthcare provider device in multiple channels, such as in an immediate manner or in a weekly aggregated format. Workflow instructions may be electronically linked by the cloud server or healthcare provider server to each tag, so that a reviewer of the data will have guidance about possible actions to be undertaken when a tag appears and an escalation of clinical review determined by the cloud server or healthcare provider server that might be appropriate.

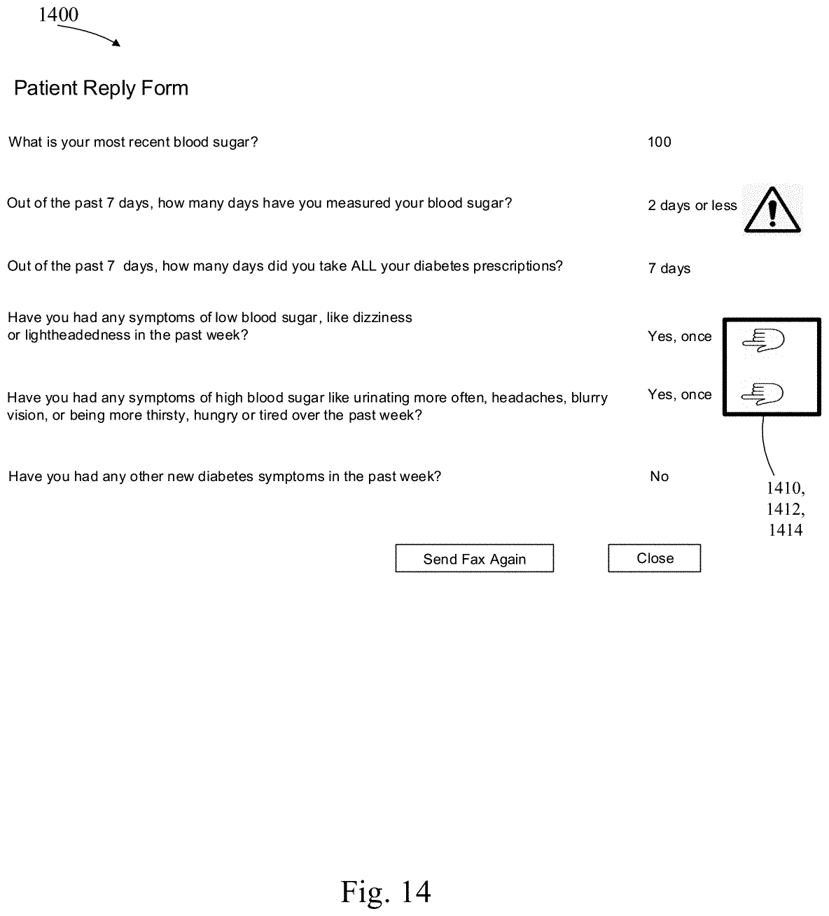

FIG. 14 depicts an example method, comprising at least one of, linking 1410 a patient device and a healthcare provider server, requesting 1412 a patient response to a message sent to the patient device including a query of a health-related issue and receiving 1414 the patient response from the patient device to the query. The patient device may be wireless or wired. The healthcare provider server may be a local server, a remote server a cloud server and the like. The data related to the healthcare related issue may be stored on the healthcare provider server or a cloud server.

The method may also include determining 1416 a content similarity of the patient response to at least one previous response, determining 1418 a content recurrence count of the patient response indicating a similar response content and determining 1420 a content recurrence timeliness of the patient response indicating the similar response content. The content similarity of the patient response may be the response from the patient device. The similarity of responses is a determination of whether a response is occurring more than once. If the response is occurring more than once, the system determines how often and when in time the recurrences happen. The recurrence information may be stored on the healthcare provider server or the cloud server. If the recurrence information is stored in the cloud server, a portion of the data may be modified to protect the identity of the patient for response pooling purposes.

The method may additionally comprise determining 1422 an urgency level of the patient response based on the health-related issue, the content recurrence count and the content recurrence timeliness, tagging 1424 the patient response as an urgent tagged response if the determined urgency level exceeds a predetermined urgency threshold of the patient for the health-related issue, the content recurrence count and the content recurrence timeliness and providing 1426 the request and the urgent tagged response to a healthcare provider device. The system looks at the response, whether the response reoccurs, the responses urgency, repetition and timeliness and makes a determination whether the situation is serious enough to immediately forward to the healthcare providers device.

The method may also include determining 1428 a set of possible reasons that the patient response triggered the urgent tagged response and presenting 1430 to the healthcare provider device a list of alternative treatment regimens in response to the urgent tagged response. The determination of the possible reasons may be based on the medicine, its initial effects on a patient, when issues may be indicative of a serious reaction and the pool of responses of similar patient's having similar demographics and taking similar medications. This pooled information may be kept in the cloud based system.

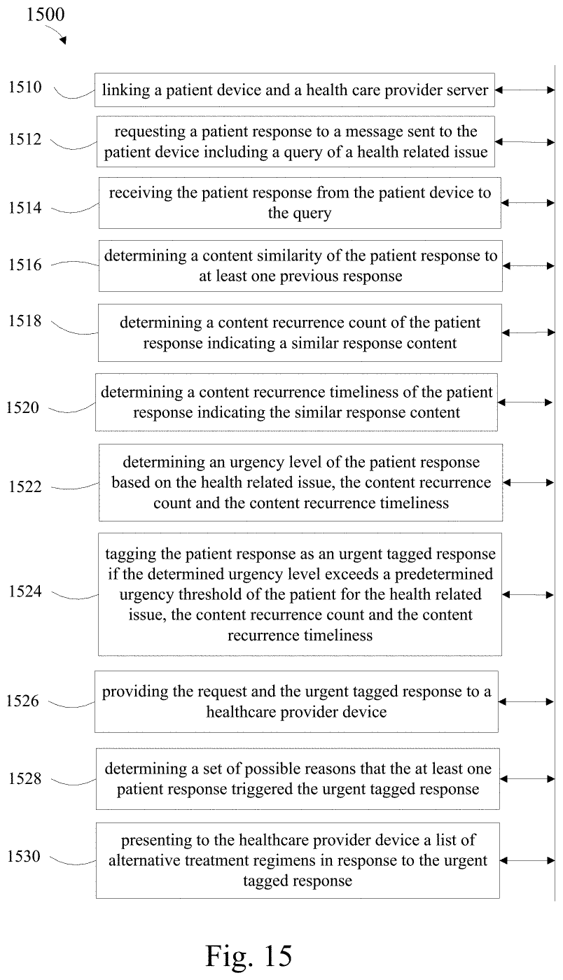

FIG. 15 depicts a non-transitory computer readable medium comprising instructions that, when read by a processor, cause the processor to perform at least one of, linking 1510 a patient device and a healthcare provider server, requesting 1512 a patient response to a message sent to the patient device including a query of a health-related issue and receiving 1514 the patient response from the patient device to the query.

The processor may additionally perform the tasks of determining 1516 a content similarity of the patient response to at least one previous response, determining 1518 a content recurrence count of the patient response indicating a similar response content and determining 1520 a content recurrence timeliness of the patient response indicating the similar response content. The processor may additionally perform determining 1522 an urgency level of the patient response based on the health-related issue and the content recurrence count and the content recurrence timeliness. The processor may perform tagging 1524 the patient response as an urgent tagged response if the determined urgency level exceeds a predetermined urgency threshold of the patient for the health-related issue, the content recurrence count and the content recurrence timeliness and providing 1526 the request and the urgent tagged response a healthcare provider device.

The processor may also perform determining 1528 a set of possible reasons that the patient response triggered the urgent tagged response and presenting 1530 to the healthcare provider device a list of alternative treatment regimens in response to the urgent tagged response.

A system, comprising, at least one cloud-based processor and at least one memory electrically coupled to the at least one cloud-based processor and storing an application, wherein the processor performs at least one operation to, link a patient device and a healthcare provider server, request a patient response to a message sent to the patient device including a query of a health-related issue and receive the patient response from the patient device to the query.

The system may also determine a content similarity of the patient response to at least one previous response, determine a content recurrence count of the patient response indicating a similar response content, determine a content recurrence timeliness of the patient response indicating the similar response content, determine an urgency level of the patient response based on the health-related issue, the content recurrence count and the content recurrence timeliness and tag the patient response as an urgent tagged response if the determined urgency level exceeds a predetermined urgency threshold of the patient for the health-related issue, the content recurrence count and the content recurrence timeliness.

The system may also provide the request and the urgent tagged response a healthcare provider device, determine a set of possible reasons that the patient response triggered the urgent tagged response and present to the healthcare provider device a list of alternative treatment regimens in response to the urgent tagged response.

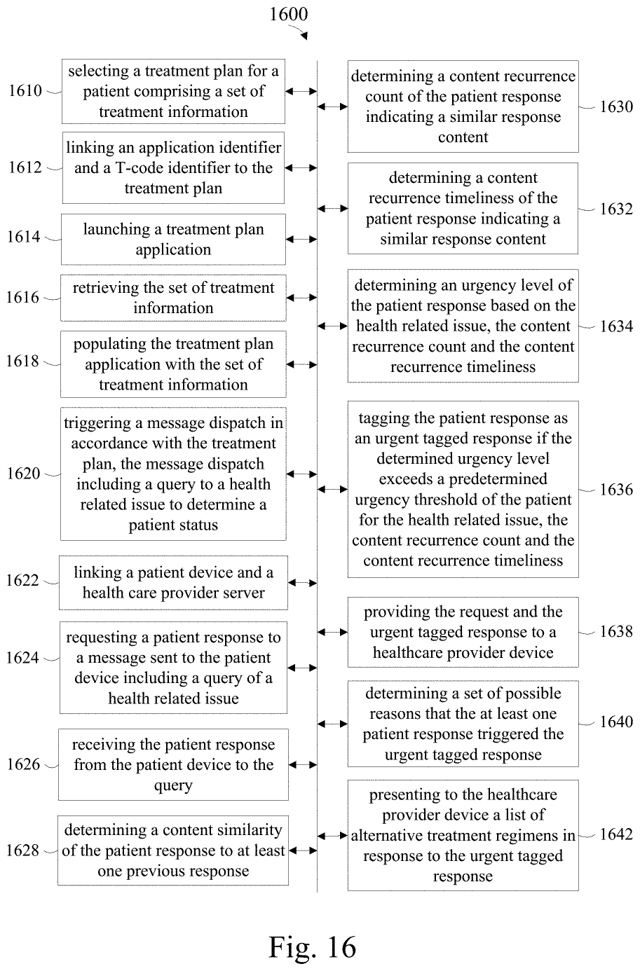

FIG. 16 depicts an example method, comprising at least one of, selecting 1610 a treatment plan for a patient comprising a set of treatment information, linking 1612 an application identifier and a T-code identifier to the treatment plan, launching 1614 a treatment plan application and retrieving 1616 the set of treatment information. The method may also include populating 1618 the treatment plan application with the set of treatment information and triggering 1620 a message dispatch in accordance with the treatment plan, the message dispatch including the query to the health-related issue to determine a patient status.

The method may also comprise linking 1622 a patient device and a healthcare provider server, requesting 1624 a patient response to a message sent to the patient device including a query of a health-related issue and receiving 1626 the patient response from the patient device to the query.

The method may also include determining 1628 a content similarity of the patient response to at least one previous response, determining 1630 a content recurrence count of the patient response indicating a similar response content, determining 1632 a content recurrence timeliness of the patient response indicating the similar response content and determining 1634 an urgency level of the patient response based on the health-related issue, the content recurrence count and the content recurrence timeliness.

The method may additionally comprise tagging 1636 the patient response as an urgent tagged response if the determined urgency level exceeds a predetermined urgency threshold of the patient for the health-related issue, the content recurrence count and the content recurrence timeliness, providing 1638 the request and the urgent tagged response to a healthcare provider device, determining 1640 a set of possible reasons that the patient response triggered the urgent tagged response and presenting 1642 to the healthcare provider device a list of alternative treatment regimens in response to the urgent tagged response.

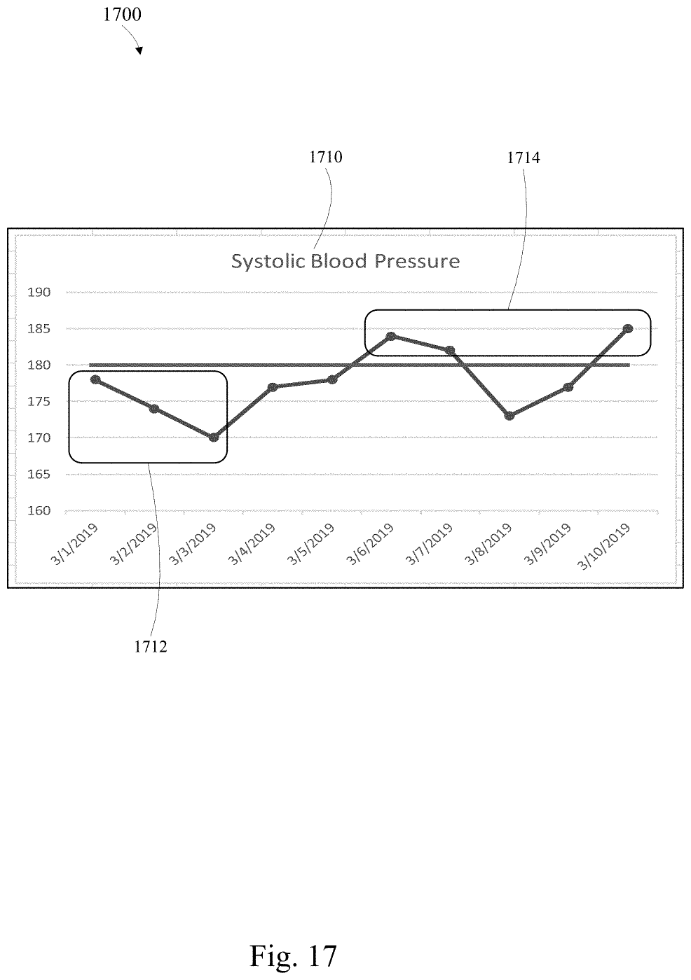

An example of a multiple warning for systolic blood pressure is depicted in FIG. 17. In this example chart the systolic blood pressure trend 1710 is depicted. The first three values shown as 1712 appear to be improving for the patient. The high warning value of 180 mm-hg of systolic pressure sets the bar against which the data is reviewed. In this example three data points 1714 exceed the warning value, and tend to indicate that the patient is experiencing an urgent issue and indicates that multiple warnings warrant immediate attention. The failure may be one of not adhering to the drug schedule, stress, high salt foods etc. One-time events may not indicate a an urgent issue, however multiple events over a specific timeframe may indicate an urgent issue for a failure to progress and may warrant immediate attention. The healthcare provider may determine the number of warnings within a specific time frame to indicate an urgent issue. In the case there is progress, the high warning value may be reduced as determined by the healthcare provider or suggested by the cloud based server.

The operations of a method or algorithm described in connection with the embodiments disclosed herein may be embodied directly in hardware, in a computer program executed by a processor, or in a combination of the two. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

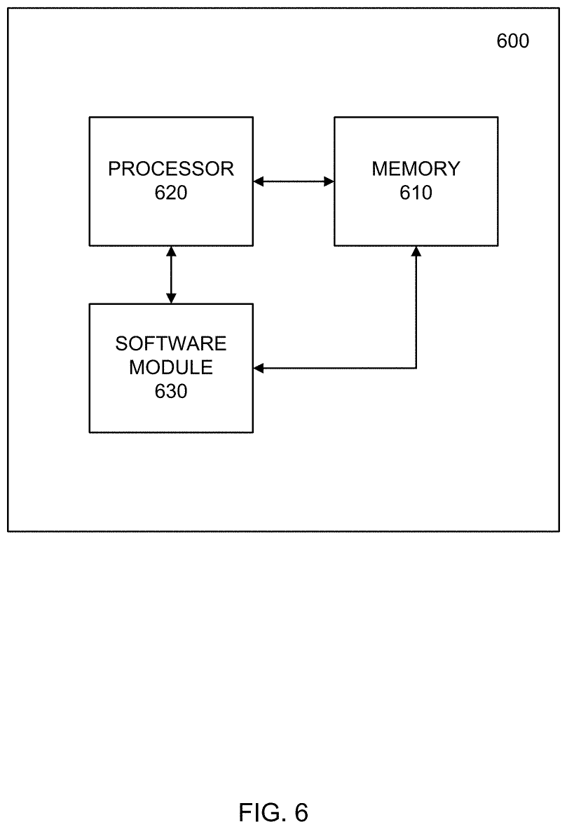

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 6 illustrates an example network element, which may represent any of the above-described network components of the other figures.

As illustrated in FIG. 6, a memory 610 and a processor 620 may be discrete components of the network entity 600 that are used to execute an application or set of operations. The application may be coded in software in a computer language understood by the processor 620, and stored in a computer readable medium, such as, the memory 610. The computer readable medium may be a non-transitory computer readable medium that includes tangible hardware components in addition to software stored in memory. Furthermore, a software module 630 may be another discrete entity that is part of the network entity 600, and which contains software instructions that may be executed by the processor 620. In addition to the above noted components of the network entity 600, the network entity 600 may also have a transmitter and receiver pair configured to receive and transmit communication signals (not shown).

Although an exemplary embodiment of the system, method, and computer readable medium of the present application has been illustrated in the accompanied drawings and described in the foregoing detailed description, it will be understood that the application is not limited to the embodiments disclosed, but is capable of numerous rearrangements, modifications, and substitutions without departing from the spirit or scope of the application as set forth and defined by the following claims. For example, the capabilities of the system of the various figures can be performed by one or more of the modules or components described herein or in a distributed architecture and may include a transmitter, receiver or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone or any other suitable computing device, or combination of devices. Presenting the above-described functions as being performed by a “system” is not intended to limit the scope of the present application in any way, but is intended to provide one example of many embodiments of the present application. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed, but is merely representative of selected embodiments of the application.

One having ordinary skill in the art will readily understand that the application as discussed above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent, while remaining within the spirit and scope of the application. In order to determine the metes and bounds of the application, therefore, reference should be made to the appended claims.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms etc.) thereto.

What is claimed is:

1. A method, comprising:
linking a patient device and a health care provider server;
requesting a patient response to a message sent to the patient device including a query of a health-related issue;
receiving the patient response from the patient device to the query;
determining a content similarity of the patient response to at least one previous response;
determining a content recurrence count of the patient response indicating a similar response content;
determining a content recurrence timeliness of the patient response indicating the similar response content;
determining an urgency level of the patient response based on the health-related issue, the content recurrence count and the content recurrence timeliness;
tagging the patient response as an urgent tagged response if the determined urgency level exceeds a predetermined urgency threshold of the patient for the health-related issue, the content recurrence count and the content recurrence timeliness;
providing the request and the urgent tagged response to a healthcare provider device; determining a set of possible reasons that the patient response triggered the urgent tagged response; and
presenting to the healthcare provider device a list of alternative treatment regimens in response to the urgent tagged response.

2. The method of claim 1 further comprising sending at least one follow-on query based on the urgent tagged response.

3. The method of claim 1 further comprising grading the urgency level of the urgent tagged response, wherein the grading is at least one of an immediate medical action advisory, a follow-up advisory and a medical history review advisory.

4. The method of claim 1 further comprising tagging the response as a non-urgent tagged response if the determined urgency level does not meet the predetermined urgency threshold of the patient for the health-related issue.

5. The method of claim 4 further comprising proposing a set of work flow instructions linked to at least one of the urgent tagged response and the non-urgent tagged response.

6. The method of claim 1 further comprising proposing a set of work flow instructions linked to the urgent tagged response.

7. The method of claim 6 further comprising presenting a clinical escalation review advisory linked to the urgent tagged response.

8. The method of claim 1 wherein the request is provided to the patient at a clinically-relevant time interval based on the health-related issue.

9. The method of claim 1, further comprising:
selecting a treatment plan for a patient comprising a set of treatment information;
linking an application identifier and a T-code identifier to the treatment plan;
launching a treatment plan application;
retrieving the set of treatment information;
populating the treatment plan application with the set of treatment information; and
triggering a message dispatch in accordance with the treatment plan, the message dispatch including the query to the health-related issue to determine a patient status.

10. A non-transitory computer readable medium comprising instructions that, when read by a processor, cause the processor to perform:
linking a patient device and a health care provider server;
requesting a patient response to a message sent to the patient device including a query of a health-related issue;
receiving the patient response from the patient device to the query;
determining a content similarity of the patient response to at least one previous response;
determining a content recurrence count of the patient response indicating a similar response content;
determining a content recurrence timeliness of the patient response indicating the similar response content;
determining an urgency level of the patient response based on the health-related issue, the content recurrence count and the content recurrence timeliness;
tagging the patient response as an urgent tagged response if the determined urgency level exceeds a predetermined urgency threshold of the patient for the health-related issue, the content recurrence count and the content recurrence timeliness;
providing the request and the urgent tagged response a healthcare provider device;
determining a set of possible reasons that the patient response triggered the urgent tagged response; and
presenting to the healthcare provider device a list of alternative treatment regimens in response to the urgent tagged response.

11. The non-transitory computer readable medium of claim 10, comprising instructions, that when read by the processor, cause the processor to perform:
selecting a treatment plan for the patient comprising a set of treatment information;
linking an application identifier and a T-code identifier to the treatment plan;
launching a treatment plan application;
retrieving the set of treatment information;
populating the treatment plan application with the set of treatment information; and
triggering a message dispatch in accordance with the treatment plan, the message dispatch including the query to the health-related issue to determine a patient status.

12. The non-transitory computer readable medium of claim 11, comprising instructions, that when read by the processor, cause the processor to perform:
loading a trigger table having a set of trigger dates based on the treatment plan, the message dispatch responsive to the set of trigger dates; and
receiving a message start date and receiving an initialization message from a patient mobile device to initiate the treatment plan and to initialize the set of trigger dates in the trigger table.

13. The non-transitory computer readable medium of claim 11, comprising instructions, that when read by the processor, cause the processor to perform at least one of creating the treatment plan and modifying the treatment plan.

14. The non-transitory computer readable medium of claim 10, comprising instructions, that when read by the processor, cause the processor to perform sending at least one follow-on query based on the urgent tagged response.

15. The non-transitory computer readable medium of claim 10, comprising instructions, that when read by the processor, cause the processor to perform grading the urgency level of the urgent tagged response, wherein the grading is at least one of an immediate medical action advisory, a follow-up advisory and a medical history review advisory.

16. The non-transitory computer readable medium of claim 10, comprising instructions, that when read by the processor, cause the processor to perform:
tagging the response as a non-urgent tagged response if the determined urgency level does not meet the predetermined urgency threshold of the patient for the health-related issue; and
proposing a set of work-flow instructions linked to at least one of the urgent tagged response and the non-urgent tagged response.

17. The non-transitory computer readable medium of claim 10, comprising instructions, that when read by the processor, cause the processor to perform:
proposing a set of work-flow instructions linked to the urgent tagged response; and
presenting a clinical escalation review advisory linked to the urgent tagged response.

18. A system, comprising:
at least one cloud-based processor; and
at least one memory electrically coupled to the at least one cloud-based processor and storing an application,
wherein the processor performs operations to:
link a patient device and a health care provider server;
request a patient response to a message sent to the patient device including a query of a health-related issue;
receive the patient response from the patient device to the query;
determine a content similarity of the patient response to at least one previous response;
determine a content recurrence count of the patient response indicating a similar response content;
determine a content recurrence timeliness of the patient response indicating the similar response content;
determine an urgency level of the patient response based on the health-related issue, the content recurrence count and the content recurrence timeliness;
tag the patient response as an urgent tagged response if the determined urgency level exceeds a predetermined urgency threshold of the patient for the health-related issue, the content recurrence count and the content recurrence timeliness;
provide the request and the urgent tagged response a healthcare provider device;
determine a set of possible reasons that the patient response triggered the urgent tagged response; and
present to the healthcare provider device a list of alternative treatment regimens in response to the urgent tagged response.

19. The system of claim 18, wherein the processor further performs an operation to:
tag the response as a non-urgent tagged response if the determined urgency level does not meet the predetermined urgency threshold of the patient for the health-related issue; and
propose a set of work-flow instructions linked to at least one of the urgent tagged response and the non-urgent tagged response.

20. The system of claim 18, wherein the processor further performs an operation to send at least one follow-on query based on the urgent tagged response.
